(19) United States
(12) Patent Application Publication
(10) Pub. No.: US 2025/0065082 A1
(43) Pub. Date: Feb. 27, 2025

(54) CATHETER

(71) Applicant: TERUMO KABUSHIKI KAISHA, Tokyo (JP)

(72) Inventors: Takashi ITO, Shizuoka-shi (JP); Mitsuki HOSHI, Irvine, CA (US); Motohiro FUNAKI, Fujinomiya-shi (JP)

(73) Assignee: TERUMO KABUSHIKI KAISHA, Tokyo (JP)

(21) Appl. No.: 18/938,831

(22) Filed: Nov. 6, 2024

Related U.S. Application Data
(63) Continuation of application No. PCT/JP2023/023166, filed on Jun. 22, 2023.

(30) Foreign Application Priority Data
Jul. 1, 2022 (JP) 2022-106836

(51) Int. Cl. A61M 25/01; A61M 25/00

(52) U.S. Cl. CPC A61M 25/0108; A61M 25/0023; A61M 25/0045; A61M 25/005; A61M 2205/32

(57) ABSTRACT

A catheter that includes a shaft having a lumen communicating from the distal end to the proximal end. The shaft has a plurality of radiopaque markers at different positions in the axial direction of the shaft. The markers include a first marker that is the distal-most marker, and a second marker located adjacent to the first marker on the proximal side. The thickness of the first marker along the radial direction of the shaft is different from the thickness of the second marker along the radial direction.

### CROSS-REFERENCES TO RELATED APPLICATIONS

This application is a continuation of International Application No. PCT/JP2023/023166 filed on Jun. 22, 2023, which claims priority to Japanese Patent Application No. 2022-106836 filed on Jul. 1, 2022, the entire content of both of which is incorporated herein by reference.

### TECHNOLOGICAL FIELD

The present disclosure generally relates to a catheter used in a lumen such as a blood vessel.

### BACKGROUND DISCUSSION

In recent years, intraluminal treatment of a blood vessel using a catheter has been actively performed because surgical invasion is very low. For example, a catheter that is selectively introduced and used into a complicated branched blood vessel in the body is generally pushed along a guide wire that is introduced into the blood vessel in advance, and a therapeutic medical agent or a diagnostic contrast agent can be circulated through a lumen. In addition, in the catheter, a radiopaque marker is disposed on a shaft so that the position in the lumen can be recognized from outside of the body (see, for example, International Patent Application Publication No. WO 2021/250980).

The thicker the marker of the catheter, the higher the visibility. However, when the marker is relatively thick, the outer diameter of the catheter tends to be large, and the passability through the blood vessel may be reduced.

### SUMMARY

A catheter that includes a plurality of markers and is capable of appropriately imparting X-ray visibility and passability through a blood vessel.

A catheter is disclosed that includes a shaft that has a lumen communicating from a distal end to a proximal end, wherein the shaft has a plurality of markers that is radiopaque at different positions in an axial direction of the shaft, the plurality of markers includes a first marker that is a distal-most marker and a second marker that is located adjacent to the first marker on a proximal side, and a thickness of the first marker along a radial direction of the shaft is different from a thickness of the second marker along the radial direction.

The catheter configured as described above has the first marker and the second marker that are different in thickness, and thus, an appropriate thickness can be individually set for each marker. Therefore, the catheter having a plurality of markers can appropriately impart X-ray visibility and passability through a blood vessel.

The thickness of the first marker along the radial direction may be greater than the thickness of the second marker along the radial direction. With this configuration, the catheter can improve X-ray visibility by increasing the thickness of the first marker located in a section on the distal side of the catheter where the importance of position recognition is high, and can improve passability of the catheter into a blood vessel while ensuring the X-ray visibility by decreasing the thickness of the second marker located in a section on the proximal side where the importance of position recognition is not as high as the section where the first marker is placed.

One of the markers may be a third marker located on the proximal side with respect to the second marker, and the thickness of the first marker along the radial direction may be greater than the thickness of the third marker along the radial direction. With this configuration, the catheter includes the third marker different from the first marker and the second marker to enable position recognition with X-ray at a plurality of locations of the shaft. Further, the catheter can improve passability through the blood vessel by setting the thicknesses of the second marker and the third marker to be smaller than the thickness of the first marker.

The thickness of the first marker along the radial direction may be smaller than the thickness of the second marker along the radial direction. With this configuration, the catheter can improve X-ray visibility by increasing the thickness of the second marker, and can improve passability through the blood vessel while ensuring the X-ray visibility at the distal portion of the catheter by decreasing the thickness of the first marker on the distal side with respect to the second marker.

One of the markers may be a third marker located on the proximal side with respect to the second marker, and the thickness of the third marker along the radial direction may be greater than the thickness of the first marker and the thickness of the second marker along the radial direction. With this configuration, the catheter includes the third marker different from the first marker and the second marker to enable position recognition by X-ray at a plurality of locations of the shaft. Further, the catheter can improve passability through the blood vessel in a wide range at the distal portion of the catheter in the axial direction by decreasing the thicknesses of the first marker and the second marker.

Another aspect for achieving the above object provides a catheter including a shaft that has a lumen communicating from a distal end to a proximal end, wherein the shaft has a plurality of markers that is radiopaque at different positions in an axial direction of the shaft, the plurality of markers includes a first marker that is a distal-most marker, and an outer diameter of a first section of the shaft where the first marker is placed is different from an outer diameter of a section of the shaft where a marker included in the plurality of markers and different from the first marker is placed.

Another aspect of the catheter configured as described above according to the present embodiment can improve X-ray visibility by increasing the thickness of the marker located in a section where the importance of position recognition is high, and can improve passability of the catheter into the blood vessel while ensuring the X-ray visibility by decreasing the thickness of the marker located in a section where the importance of position recognition is not so high.

The plurality of markers may include a second marker that is located adjacent to the first marker on the proximal side, and the outer diameter of the first section may be greater than an outer diameter of a second section of the shaft where the second marker is placed. With this configuration, the catheter can improve X-ray visibility by increasing the thickness of the first marker located in the first section on the distal side where the importance of position recognition is high, and can improve passability of the catheter into the blood vessel while ensuring the X-ray visibility by decreasing the thickness of the second marker that is located in the second section on the proximal side where the importance of position recognition is not as high as the first marker.

One of the markers may be a third marker located on the proximal side with respect to the second marker, and the outer diameter of the first section may be greater than an outer diameter of a third section of the shaft where the third marker is placed. With this configuration, the catheter can improve X-ray visibility by increasing the thickness of the first marker located in the first section on the distal side where the importance of position recognition is high, and can improve passability of the catheter into the blood vessel while ensuring the X-ray visibility by decreasing the thickness of the third marker located in the third section on the proximal side where the importance of position recognition is not as high as the first marker.

A catheter is disclosed comprising: a shaft that has a lumen communicating from a distal end to a proximal end, the shaft having a plurality of markers that is radiopaque at different positions in an axial direction of the shaft; the plurality of markers including a first marker that is a distal-most marker and a second marker that is located adjacent to the first marker on a proximal side; a thickness of the first marker along a radial direction of the shaft is different from a thickness of the second marker along the radial direction; and an outer diameter of a first section of the shaft where the first marker is placed is different from an outer diameter of a section of the shaft where the second marker included in the plurality of markers is placed.

### BRIEF DESCRIPTION OF THE DRAWINGS

FIG. 1 is a plan view illustrating a catheter according to a first embodiment.

FIG. 2 is a cross-sectional view illustrating a distal portion of the catheter according to the first embodiment.

FIG. 3 is a cross-sectional view illustrating a distal portion of a catheter according to a modification of the first embodiment.

FIG. 4 is a cross-sectional view illustrating a distal portion of a catheter according to a second embodiment.

FIG. 5 is a cross-sectional view illustrating a distal portion of a catheter according to a modification of the second embodiment.

FIG. 6 is a cross-sectional view illustrating a distal portion of a catheter according to a third embodiment.

FIG. 7 is a cross-sectional view illustrating a distal portion of a catheter according to a modification of the third embodiment.

FIG. 8 is a cross-sectional view illustrating a distal portion of a catheter according to another modification.

### DETAILED DESCRIPTION

Set forth below with reference to the accompanying drawings is a detailed description of embodiments of a catheter used in a lumen such as a blood vessel. Note that the dimensions in the drawings may be exaggerated and different from actual dimensions for convenience of description. In the present specification and the drawings, components having substantially the same functional configuration are denoted by the same reference numerals, and the redundant description will not be repeated. In the present specification, a side of a catheter to be inserted into a body lumen is referred to as a “distal side”, and a side to be operated is referred to as a “proximal side”.

### First Embodiment

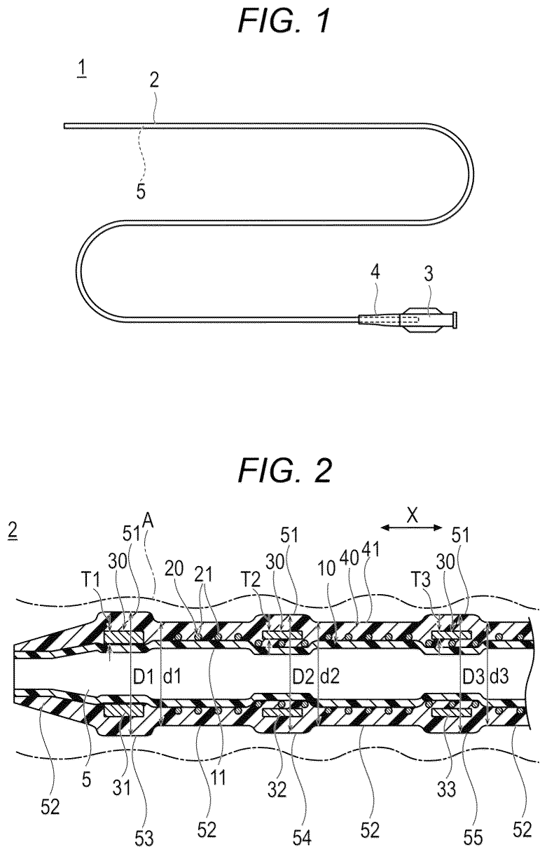

A catheter 1 according to the first embodiment is introduced into a blood vessel from a radial artery of an arm, a femoral artery of a foot, or a peripheral artery of a lower limb into an artery of a lower limb, and can be used for treatment, diagnosis, or the like. The catheter 1 can be, for example, the support catheter 1, but may be a device for other purposes. The artery of the lower limb is an artery near the aortoiliac bifurcation and an artery distal to the aortoiliac bifurcation. As illustrated in FIG. 1, the catheter 1 includes an elongated shaft 2, a hub 3 connected to a proximal end of the shaft 2, and a kink protector 4 provided at a connection portion between the shaft 2 and the hub 3.

As illustrated in FIGS. 1 and 2, the shaft 2 is a tubular member having flexibility, and has a lumen 5 formed inside from the proximal end to the distal end. A guide wire is inserted through the lumen 5 when the catheter 1 is inserted into a blood vessel. In addition, the lumen 5 can also be used as a passage for medicinal solutions, embolic materials, contrast agents, medical instruments, or the like.

The effective length of the shaft 2 is not particularly limited and can be appropriately set depending on the blood vessel into which the shaft 2 is to be inserted. For example, the effective length of the shaft 2 can be 200 mm to 2600 mm. Note that the effective length of the shaft 2 is a length of a section that can be inserted into a blood vessel, a sheath, or the like. In the present embodiment, the effective length is a length from the distal-most end of the kink protector 4 to the distal-most end of the shaft 2.

The shaft 2 has a plurality of layers, and includes an inner layer 10 defining an inner surface 11 of the lumen 5, a reinforcing body 20 disposed on the outer side of the inner layer 10 in the radial direction, a plurality of markers 30 disposed outside the inner layer 10 or the reinforcing body 20 in the radial direction, and an outer layer 40 formed on the outer side of the inner layer 10, the reinforcing body 20, and the markers 30 in the radial direction. At least a part of the markers 30 may be embedded in the outer layer 40. The radial direction coincides with the radial direction from the axial center of the shaft 2. The outer side in the radial direction is a side away from the axial center of the shaft 2 in the radial direction.

The inner diameter of the inner layer 10 is not particularly limited, and is preferably 0.2 mm to 1.2 mm. The inner diameter and the outer diameter of the inner layer 10 at positions covered with the markers 30 are smaller than those at sections adjacent to the positions in an axial direction X of the shaft 2.

The lumen 5 is formed inside the inner layer 10. As a material of the inner layer 10, a thermoplastic resin, a thermosetting resin, or the like can be applied, and a fluorine-based resin such as polytetrafluoroethylene (PTFE), a low-friction material such as high-density polyethylene (HDPE), or the like is preferable. In addition, a polyamide resin, a polyamide elastomer, polyester, a polyester elastomer, or the like may be used.

Each of the markers 30 is a radiopaque (radiographic contrast) material disposed on the outside of the inner layer 10 in the radial direction. The plurality of markers 30 can include, for example, a first marker 31, a second marker 32, and a third marker 33. The number of markers 30 may be two or four or more, but is preferably less than five. The first marker 31 is the marker 30 on the distal-most side in the axial direction X. The second marker 32 is the marker 30 disposed adjacent to the first marker 31 on the proximal side with respect to the first marker 31. The third marker 33 is the marker 30 disposed adjacent to the second marker 32 on the proximal side with respect to the second marker 32. The markers 30 are disposed apart from each other by a predetermined length in the axial direction X. Each of the markers 30 is a member formed by covering a member formed from a radiopaque material and having a C-shaped cross section orthogonal to the axial direction X on the reinforcing body 20 outside of the inner layer 10, and then, crimping the covered member into a tubular shape. However, each of the markers 30 is not limited to a member formed by covering the member formed from the radiopaque material and having a C-shaped cross section orthogonal to the axial direction C on the reinforcing body 20 outside of the inner layer 10, and then, crimping the covered member into the tubular shape. For example, each of the markers 30 may be formed by winding a wire or a belt-shaped member made of a radiopaque material in a coil shape, or may be a tube or tubular member.

A thickness T1 of the first marker 31 along the radial direction of the shaft 2 is greater than a thickness T2 of the second marker 32 along the radial direction of the shaft 2 and greater than a thickness T3 of the third marker 33 along the radial direction of the shaft 2.

The thicknesses T1, T2, and T3 of the markers 30 are not particularly limited, but are preferably, for example, 0.005 mm to 0.1 mm, more preferably 0.01 mm to 0.08 mm, still more preferably 0.02 mm to 0.04 mm. As an example, the thickness T1 of the first marker 31 is 0.03 mm, and the thickness T2 of the second marker 32 and the thickness T3 of the third marker 33 are 0.025 mm.

The length of each marker 30 in the axial direction X is not particularly limited, and can be, for example, 0.5 mm to 5 mm.

Examples of a material that can be preferably used for the marker 30 can include a material obtained by kneading an X-ray contrast agent such as: platinum, gold, silver, tungsten, iridium, molybdenum, tantalum, an alloy thereof, or a metal powder thereof; or barium sulfate, bismuth oxide, or a coupling compound thereof. Besides, a metal coil or solder having contrast property may be used. As an example, a platinum-iridium alloy is used.

The first marker 31 on the distal-most side preferably has high visibility with X-ray imaging. When the visibility of the first marker 31 with X-ray imaging is high, the visibility of the second marker 32 and the third marker 33 with X-ray imaging may be lower as long as they are visible. Thus, the thicknesses T2 and T3 of the second marker 32 and the third marker 33 may be smaller than the thickness T1 of the first marker 31, and the materials of the second marker 32 and the third marker 33 may be less radiopaque than the material of the first marker 31.

The reinforcing body 20 is formed by braiding a plurality of wire materials 21 in a tubular shape to have a gap on the outer periphery of the inner layer 10. In the reinforcing body 20, the wire material 21 may be horizontally wound in the same direction or wound with the winding direction being changed such as being wound clockwise or counterclockwise. Further, the winding pitch, the lattice distance, the inclination angle with respect to the circumferential direction, or the like may be changed depending on the position. That is, the configuration is not particularly limited. For example, one wire material 21 may be wound in a coil shape in one direction.

The reinforcing body 20 may be disposed over the entire length of the shaft 2 in the axial direction X, or may be disposed only in a part of the shaft 2. In the present embodiment, the distal end of the reinforcing body 20 is disposed between the first marker 31 and the second marker 32 on the proximal side with respect to the first marker 31. Accordingly, the reinforcing body 20 does not overlap the first marker 31 but overlaps the second marker 32 and the third marker 33 in the axial direction X. The section of the shaft 2 at the distal portion where the reinforcing body 20 is not disposed has relatively high flexibility, and thus, the bending direction along the guide wire can be easily changed. Further, it is possible to reduce the burden on a living tissue with which the section is to come in contact.

The wire diameter (diameter) of the wire material 21 of the reinforcing body 20 is not particularly limited, and can be, for example, 0.03 mm to 0.08 mm. As an example, the wire diameter of the wire material 21 of the reinforcing body 20 is 0.03 mm.

As the wire material 21 used for the reinforcing body 20, a metal wire such as a stainless steel wire, a platinum (Pt) wire, or a tungsten (W) wire, a resin fiber, a carbon fiber, a glass fiber, or the like can be applied, or a plurality of these wire materials 21 may be used in combination. The wire material 21 may be a round wire, an elliptic wire, or a flat wire. Each wire material 21 may be used as one line as a braid, or two or more wire materials 21 may be bundled and used as one line as a braid. For example, two wire materials 21 may be used.

The outer layer 40 is a tubular body disposed around the outer periphery of the inner layer 10, the markers 30, and the reinforcing body 20. The outer diameter of the outer layer 40 is not particularly limited, but can be, for example, preferably 0.6 m to 1.6 mm, and as an example, a marker placement portion 51 is 0.76 mm, and a marker-absent portion 52 is 0.75 mm.

Examples of a material that can be used for the outer layer 40 can include a polymer material such as polyolefin (for example, polyethylene, polypropylene, polybutene, an ethylene-propylene copolymer, an ethylene-vinyl acetate copolymer, an ionomer, or a mixture of two or more types of polymers), polyvinyl chloride, polyamide, a polyester elastomer, a polyamide elastomer, polyurethane, a polyurethane elastomer, polyimide, or a fluororesin, a thermoplastic resin such as a mixture of these, and a thermosetting resin such as an epoxy resin. The outer layer 40 may be mixed with a radiopaque substance, and the material of the outer layer 40 may be the same as the material of the inner layer 10. As an example, a resin and a radiopaque substance may be mixed.

The shaft 2 includes a plurality of marker placement portions 51 in which the markers 30 are placed and a plurality of marker-absent portions 52 in which the markers 30 are not placed in the axial direction X. The marker placement portions 51 and the marker-absent portions 52 are alternately arranged in the axial direction X.

The inner diameter of each of the marker placement portions 51 is smaller than the inner diameter of the marker-absent portion 52. Thus, it is possible to prevent a situation in which the strength of the shaft 2 decreases due to a decrease in thickness of the inner layer 10 or the outer layer 40 more than necessary because of the markers 30 being disposed. In addition, the difference between the inner diameter of the distal portion of the shaft 2 where the marker placement portion 51 is provided and the outer diameter of the guide wire passing through the lumen 5 is reduced, whereby the guide wire can be effectively held. Note that the inner diameter of the marker-absent portion 52 in the vicinity of the distal-most end of the shaft 2 is smaller than the inner diameter of the marker placement portion 51. As an example, the marker placement portion 51 is 0.44 mm, and the marker-absent portion 52 is 0.48 mm.

An outer diameter D1 of the first marker placement portion 53 where the first marker 31 is placed is greater than an outer diameter d1 of the marker-absent portion 52 adjacent to the first marker placement portion 53. The outer diameter D1 of the first marker placement portion 53 may be a maximum outer diameter of the first marker placement portion 53, an outer diameter of the first marker placement portion 53 at the central part in the axial direction X, or an average value of outer diameters of the first marker placement portion 53 at a plurality of locations in the axial direction X. Alternatively, the outer diameter d1 of the marker-absent portion 52 adjacent to the first marker placement portion 53 may be, for example, the minimum outer diameter of the marker-absent portion 52 on the proximal side with respect to the first marker placement portion 53, or may be the outer diameter of a section, which is adjacent to the first marker placement portion 53, of the marker-absent portion 52 on the proximal side with respect to the first marker placement portion 53. Alternatively, the outer diameter d1 of the marker-absent portion 52 adjacent to the first marker placement portion 53 may be the minimum outer diameter of the marker-absent portion 52 on the distal side with respect to the first marker placement portion 53, or may be the outer diameter of a section, which is adjacent to the first marker placement portion 53, of the marker-absent portion 52 on the distal side with respect to the first marker placement portion 53. Alternatively, the outer diameter d1 of the marker-absent portion 52 adjacent to the first marker placement portion 53 may be an average value of the outer diameter (or the minimum outer diameter) of the section, which is adjacent to the first marker placement portion 53, of the marker-absent portion 52 on the distal side with respect to the first marker placement portion 53 and the outer diameter (or the minimum outer diameter) of the section, which is adjacent to the first marker placement portion 53, of the marker-absent portion 52 on the proximal side with respect to the first marker placement portion 53.

An amount of protrusion of the first marker placement portion 53 from the marker-absent portion 52 to the outside in the radial direction is equal to or less than the thickness T1 of the first marker 31 as represented in the following Expression (1). The amount of protrusion of the first marker placement portion 53 from the marker-absent portion 52 to the outside in the radial direction may be less than the thickness T1 of the first marker 31. As a result, the outer diameter of the first marker placement portion 53 is reduced, and a decrease in the passability of the first marker placement portion 53 through the blood vessel A can be suppressed.

$$D 1 - d 1 \leq 2 \times T 1 \qquad Expression ( 1 )$$

An outer diameter D2 of the second marker placement portion 54 where the second marker 32 is placed is greater than an outer diameter d2 of the marker-absent portion 52 adjacent to the second marker placement portion 54. The outer diameter D2 of the second marker placement portion 54 may be a maximum outer diameter of the second marker placement portion 54, an outer diameter of the second marker placement portion 54 at the central part in the axial direction X, or an average value of outer diameters of the second marker placement portion 54 at a plurality of locations in the axial direction X. The outer diameter d2 of the marker-absent portion 52 adjacent to the second marker placement portion 54 may be, for example, the minimum outer diameter of the marker-absent portion 52 on the proximal side with respect to the second marker placement portion 54, or may be the outer diameter of a section, which is adjacent to the second marker placement portion 54, of the marker-absent portion 52 on the proximal side with respect to the second marker placement portion 54. Alternatively, the outer diameter d2 of the marker-absent portion 52 adjacent to the second marker placement portion 54 may be the minimum outer diameter of the marker-absent portion 52 on the distal side with respect to the second marker placement portion 54, or may be the outer diameter of a section, which is adjacent to the second marker placement portion 54, of the marker-absent portion 52 on the distal side with respect to the second marker placement portion 54. Alternatively, the outer diameter d2 of the marker-absent portion 52 adjacent to the second marker placement portion 54 may be an average value of the outer diameter (or the minimum outer diameter) of the section, which is adjacent to the second marker placement portion 54, of the marker-absent portion 52 on the distal side with respect to the second marker placement portion 54 and the outer diameter (or the minimum outer diameter) of the section, which is adjacent to the second marker placement portion 54, of the marker-absent portion 52 on the proximal side with respect to the second marker placement portion 54.

An amount of protrusion of the second marker placement portion 54 from the marker-absent portion 52 to the outside in the radial direction is equal to or less than the thickness T2 of the second marker 32 as represented in the following Expression (2). The amount of protrusion of the second marker placement portion 54 from the marker-absent portion 52 to the outside in the radial direction may be less than the thickness T2 of the second marker 32. As a result, the outer diameter of the second marker placement portion 54 is reduced, and a decrease in the passability of the second marker placement portion 54 through the blood vessel A can be suppressed.

$$D 2 - d 2 \leq 2 \times T 2 \qquad Expression ( 2 )$$

An outer diameter D3 of the third marker placement portion 55 where the third marker 33 is placed is greater than an outer diameter d3 of the marker-absent portion 52 adjacent to the third marker placement portion 55. The outer diameter D3 of the third marker placement portion 55 may be a maximum outer diameter of the third marker placement portion 55, an outer diameter of the third marker placement portion 55 at the central part in the axial direction X, or an average value of outer diameters of the third marker placement portion 55 at a plurality of locations in the axial direction X. The outer diameter d3 of the marker-absent portion 52 adjacent to the third marker placement portion 55 may be, for example, the minimum outer diameter of the marker-absent portion 52 on the proximal side with respect to the third marker placement portion 55, or may be the outer diameter of a section, which is adjacent to the third marker placement portion 55, of the marker-absent portion 52 on the proximal side with respect to the third marker placement portion 55. Alternatively, the outer diameter d3 of the marker-absent portion 52 adjacent to the third marker placement portion 55 may be the minimum outer diameter of the marker-absent portion 52 on the distal side with respect to the third marker placement portion 55, or may be the outer diameter of a section, which is adjacent to the third marker placement portion 55, of the marker-absent portion 52 on the distal side with respect to the third marker placement portion 55. Alternatively, the outer diameter d3 of the marker-absent portion 52 adjacent to the third marker placement portion 55 may be an average value of the outer diameter (or the minimum outer diameter) of the section, which is adjacent to the third marker placement portion 55, of the marker-absent portion 52 on the distal side with respect to the third marker placement portion 55 and the outer diameter (or the minimum outer diameter) of the section, which is adjacent to the third marker placement portion 55, of the marker-absent portion 52 on the proximal side with respect to the third marker placement portion 55.

An amount of protrusion of the third marker placement portion 55 from the marker-absent portion 52 to the outside in the radial direction is equal to or less than the thickness T3 of the third marker 33 as represented in the following Expression (3). The amount of protrusion of the third marker placement portion 55 from the marker-absent portion 52 to the outside in the radial direction may be less than the thickness T3 of the third marker 33. As a result, the outer diameter of the third marker placement portion 55 is reduced, and a decrease in the passability of the third marker placement portion 55 through the blood vessel A can be suppressed.

$$D 3 - d 3 \leq 2 \times T 3 \qquad Expression ( 3 )$$

Further, as represented in the following Expression (4), the thickness T1 of the first marker 31 is greater than the thickness T2 of the second marker 32 and the thickness T3 of the third marker 33, and the thickness T2 of the second marker 32 and the thickness T3 of the third marker 33 are equal to each other. Note that the thickness T2 of the second marker 32 and the thickness T3 of the third marker 33 may be different. Thus, the outer diameter D1 of the first marker placement portion 53 can be set to be greater than the outer diameter D2 of the second marker placement portion 54 and the outer diameter D3 of the third marker placement portion 55.

$$T 1 > T 2 = T 3 \qquad Expression ( 4 )$$

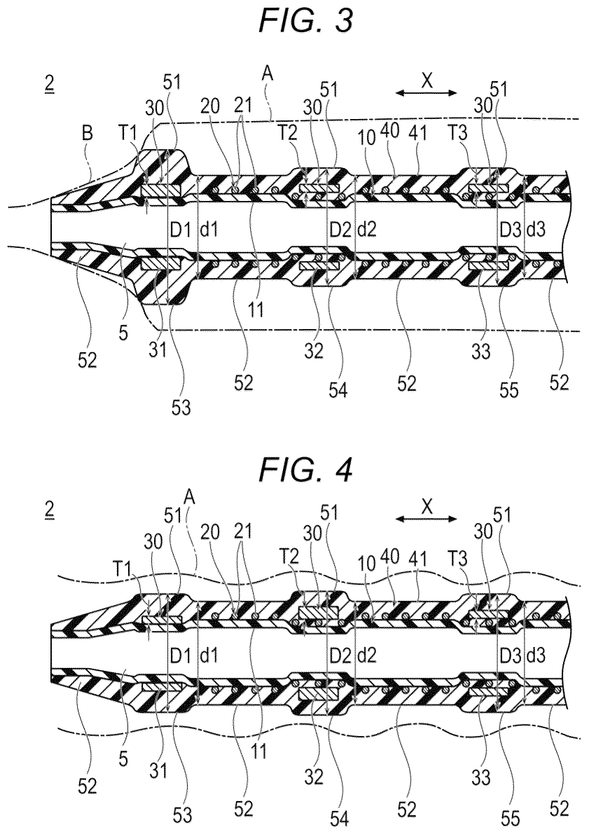

In a modification of the catheter 1 according to the first embodiment illustrated in FIG. 3, an amount of protrusion of the first marker placement portion 53 from the marker-absent portion 52 to the outside in the radial direction may exceed the thickness T1 of the first marker 31 as represented in the following Expression (5). As a result, the outer diameter D1 of the first marker placement portion 53 increases, and thus, a hard lesion B can be dug or can be widened to enable insertion.

$$D 1 - d 1 > 2 \times T 1 \qquad Expression ( 5 )$$

The outer diameter (d1, d2, d3) of the marker-absent portion 52 adjacent to the marker placement portion 51 is not particularly limited, but can be, for example, preferably 0.7 mm to 1.6 mm, more preferably 1.3 mm to 1.5 mm, still more preferably 1.35 mm to 1.45 mm. In the present embodiment, it is about 1.40 mm. The outer diameter difference ((D1−d1), (D2−d2), and (D3−d3)) which is a difference between the outer diameter of the marker placement portion 51 and the outer diameter of the marker-absent portion 52 can be, for example, preferably 0 mm to 0.06 mm, more preferably 0 mm to 0.03 mm, still more preferably 0 mm to 0.01 mm. In the present embodiment, the outer diameter difference is about 0.005 mm.

The proximal portion of the shaft 2 is liquid-tightly fixed to the hub 3 with an adhesive, by heat fusion, or with a stopper, for example. The hub 3 can function as, for example, an insertion port for the guide wire or a medical instrument into the lumen 5, or an injection port for a medicinal solution, an embolic material, a contrast agent, or the like into the lumen 5, and also functions as a grip portion when the catheter 1 is operated. The material of the hub 3 is not particularly limited, and examples of materials for the hub 3 that can be preferably used include a thermoplastic resins such as polycarbonate, polyamide, polysulfone, polyarylate, or a methacrylate-butylene-styrene copolymer.

The kink protector 4 is made of an elastic material provided to surround the periphery of the shaft 2, and suppresses kink of the shaft 2 at a connection portion between the shaft 2 and the hub 3. Examples of materials that can be preferably used for the kink protector 4 include natural rubber, silicone resin, polyester elastomer, polyamide elastomer, and polyurethane elastomer.

As described above, the catheter 1 according to the present embodiment includes a shaft 2 that has a lumen 5 communicating from the distal end to the proximal end, in which the shaft 2 has a plurality of markers 30 that is radiopaque at different positions in the axial direction X of the shaft 2, the plurality of markers 30 includes a first marker 31 that is a distal-most marker and a second marker 32 that is located adjacent to the first marker 31 on a proximal side, and a thickness T1 of the first marker 31 along a radial direction of the shaft 2 is different from a thickness T2 of the second marker 32 along the radial direction.

With this configuration, the catheter 1 has the first marker 31 and the second marker 32 that are different in thickness, and thus, an appropriate thickness can be individually set for each of the markers 30. Therefore, the catheter 1 having a plurality of markers 30 can appropriately impart X-ray visibility and passability through the blood vessel A.

In addition, the thickness of the first marker 31 along the radial direction is greater than the thickness of the second marker 32 along the radial direction. With this configuration, the catheter 1 can improve X-ray visibility by increasing the thickness of the first marker 31 located in the section on the distal side of the catheter 1 where the importance of position recognition is relatively high, and can improve passability of the catheter 1 into the blood vessel A while ensuring the X-ray visibility by decreasing the thickness of the second marker 32 located in the section on the proximal side where the importance of position recognition is not as high as the section where the first marker 31 is placed.

In addition, one of the markers 30 may be a third marker 33 located on the proximal side with respect to the second marker 32, and the thickness of the first marker 31 along the radial direction may be greater than the thickness of the third marker 33 along the radial direction. With this configuration, the catheter 1 includes the third marker 33 different from the first marker 31 and the second marker 32 to enable position recognition with X-ray at a plurality of locations of the shaft 2. Further, the catheter 1 can improve passability through the blood vessel A by setting the thicknesses of the second marker 32 and the third marker 33 to be smaller than the thickness of the first marker 31.

In addition, the catheter 1 according to the present embodiment includes a shaft 2 that has a lumen 5 communicating from a distal end to a proximal end, in which the shaft 2 has a plurality of markers 30 that is radiopaque at different positions in an axial direction X of the shaft 2, the plurality of markers 30 includes a first marker 31 that is a distal-most marker, and an outer diameter D1 of a first section (first marker placement portion 53) of the shaft 2 where the first marker 31 is placed is different from an outer diameter D2 or D3 of a section (second marker placement portion 54 or third marker placement portion 55) of the shaft 2 where a marker 30 (second marker 32 or third marker 33) included in the plurality of markers 30 and different from the first marker 31 is placed. With this configuration, the catheter 1 according to the present embodiment can improve X-ray visibility by increasing the thickness of the marker located in the section where the importance of position recognition is high, and can improve passability of the catheter 1 into the blood vessel A while ensuring the X-ray visibility by decreasing the thickness of the marker located in the section where the importance of position recognition is not so high.

In addition, the marker 30 adjacent to the first marker 31 on the proximal side is the second marker 32, and the outer diameter D1 of the first section is greater than the outer diameter D2 of the second section of the shaft 2 where the second marker 32 is placed. With this configuration, the catheter 1 can improve X-ray visibility by increasing the thickness of the first marker 31 located in the first section on the distal side where the importance of position recognition is high, and can improve passability of the catheter 1 into the blood vessel A while ensuring the X-ray visibility by decreasing the thickness of the second marker 32 that is located in the second section on the proximal side where the importance of position recognition is not as high as the first marker 31.

In addition, one of the markers 30 is a third marker 33 located on the proximal side with respect to the second marker 32, and the outer diameter D1 of the first section (first marker placement portion 53) is greater than the outer diameter D3 of the third section (third marker placement portion 55) of the shaft 2 where the third marker 33 is placed. With this configuration, the catheter 1 can improve X-ray visibility by increasing the thickness of the first marker 31 located in the first section on the distal side where the importance of position recognition is high, and can improve passability of the catheter 1 into the blood vessel A while ensuring the X-ray visibility by decreasing the thickness of the third marker 33 located in the third section on the proximal side where the importance of position recognition is not as high as the first marker 31.

### Second Embodiment

A catheter 1 according to the second embodiment illustrated in FIG. 4 is different from the first embodiment in that a thickness T2 of a second marker 32 is greater than a thickness T1 of a first marker 31 and a thickness T3 of a third marker 33 as represented by the following Expression (6). The thickness T1 of the first marker 31 and the thickness T3 of the third marker 33 are equal to each other. Note that the thickness T1 of the first marker 31 and the thickness T3 of the third marker 33 may be different. An outer diameter D2 of a second marker placement portion 54 can be greater than an outer diameter D1 of a first marker placement portion 53 and an outer diameter D3 of a third marker placement portion 55.

$$T 2 > T 1 = T 3 \qquad Expression ( 6 )$$

Therefore, the thickness T1 of the first marker 31 along the radial direction is smaller than the thickness T2 of the second marker 32 along the radial direction. With this configuration, the catheter 1 can improve X-ray visibility by increasing the thickness of the second marker 32, and can improve passability through the blood vessel A while ensuring the X-ray visibility at the distal portion (section having the first marker 31) of the catheter 1 by decreasing the thickness of the first marker 31 on the distal side with respect to the second marker 32. Then, the distal portion of the catheter 1 can be pushed into and placed in a narrowed site of the blood vessel A, whereby the catheter 1 can be easily held stably with respect to the blood vessel A, and a backup force for holding the guide wire or the like passing through the catheter 1 can be increased.

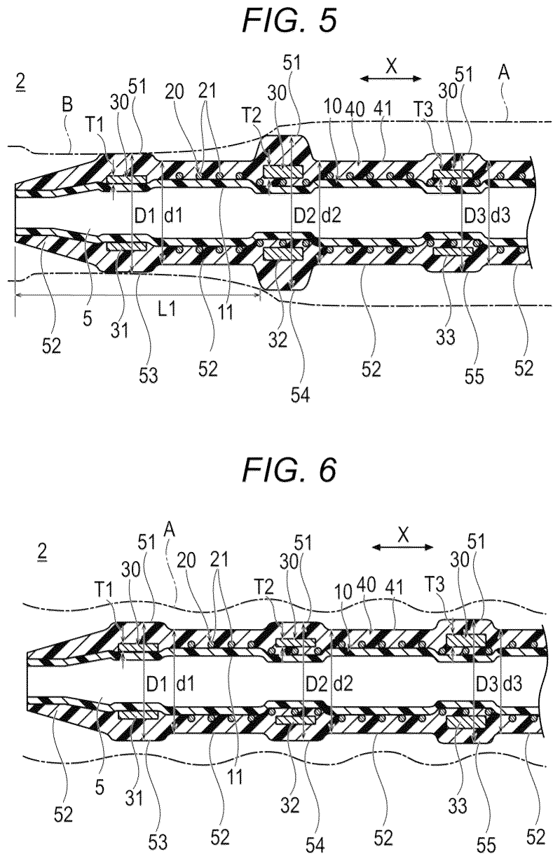

In a modification of the catheter 1 according to the second embodiment illustrated in FIG. 5, an amount of protrusion of the second marker placement portion 54 from the marker-absent portion 52 to the outside in the radial direction exceeds the thickness T2 of the second marker 32 as represented in the following Expression (7). With this configuration, the outer diameter D2 of the second marker placement portion 54 increases, and thus, a hard lesion B can be dug by the second marker placement portion 54 or widened to enable insertion by the second marker placement portion 54.

$$D 2 - d 2 > 2 \times T 2 \qquad Expression ( 7 )$$

A length L in the axial direction X from the distal end of the second marker placement portion 54 to the distal-most end of the shaft 2 is, for example, about 40 mm.

With this configuration, the second marker placement portion 54 having a large outer diameter pushes the first marker placement portion 53 with a spear-like shape that has an outer diameter not as large as that of the second marker placement portion 54 but locally widens into the short lesion B of about 40 mm, so that the distal end of the catheter 1 can be inserted so as to cut or widen the hard lesion B.

### Third Embodiment

A catheter 1 according to the third embodiment illustrated in FIG. 6 is different from the first and second embodiments in that a thickness T3 of a third marker 33 is greater than a thickness T1 of a first marker 31 and a thickness T2 of a second marker 32 as represented by the following Expression (8). The thickness T1 of the first marker 31 and the thickness T2 of the second marker 32 are equal to each other. Note that the thickness T1 of the first marker 31 and the thickness T2 of the second marker 32 may be different. An outer diameter D3 of a third marker placement portion 55 can be greater than an outer diameter D1 of a first marker placement portion 53 and an outer diameter D2 of a second marker placement portion 54.

$$T 3 > T 1 = T 2 \qquad Expression ( 8 )$$

Therefore, one of the markers 30 is a third marker 33 located on the proximal side with respect to the second marker 32, and the thickness T3 of the third marker 33 along the radial direction is greater than the thickness T1 of the first marker 31 and the thickness T2 of the second marker 32 along the radial direction. With this configuration, the catheter 1 includes the third marker 33 different from the first marker 31 and the second marker 32 to enable position recognition by X-ray at a plurality of locations of the shaft 2. Further, the catheter 1 can improve passability through the blood vessel A in a wide range (region having the first marker 31 and the second marker 32) at the distal portion of the catheter 1 in the axial direction X by decreasing the thicknesses of the first marker 31 and the second marker 32. Then, a wide range of the distal portion of the catheter 1 in the axial direction X can be pushed into and placed in a narrowed site of the blood vessel A, whereby the catheter 1 can be easily held stably with respect to the blood vessel A, and a backup force for holding the guide wire or the like passing through the catheter 1 can be increased.

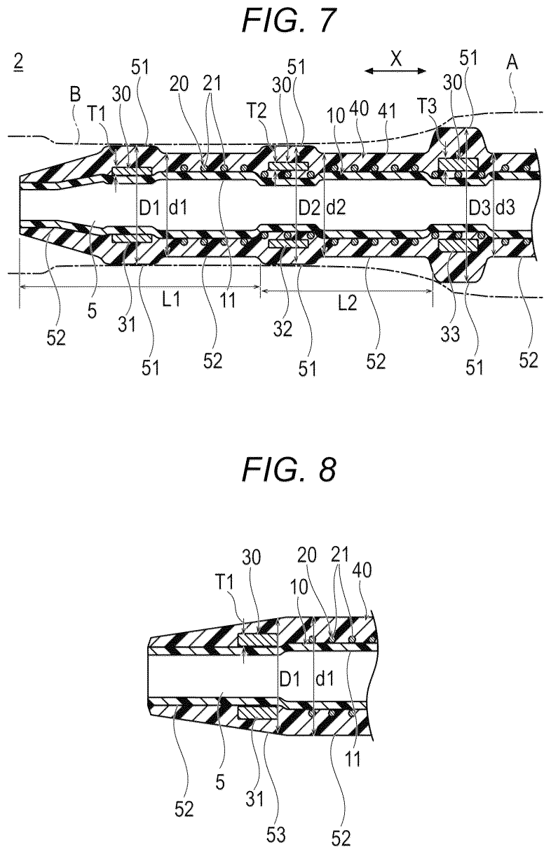

In a modification of the catheter 1 according to the third embodiment illustrated in FIG. 7, an amount of protrusion of the third marker placement portion 55 from the marker-absent portion 52 to the outside in the radial direction exceeds the thickness T3 of the third marker 33 as represented in the following Expression (9). Thus, the outer diameter D3 of the third marker placement portion 55 increases, and thus, a hard lesion B can be broken up by the third marker placement portion 55 or widened to enable insertion by the third marker placement portion 55.

$$D 3 - d 3 > 2 \times T 3 \qquad Expression ( 9 )$$

A length L1 in the axial direction X from the distal end of the second marker placement portion 54 to the distal-most end of the shaft 2 is, for example, about 40 mm, and a length L2 in the axial direction X from the distal end of the third marker placement portion 55 to the distal end of the second marker placement portion 54 is, for example, about 60 mm.

Thus, the section on the distal side with respect to the third marker placement portion 55 having a large outer diameter can be pushed into the long lesion B of about 100 mm. At this time, the distal end of the catheter 1 can be inserted so as to cut or widen the hard and long lesion B by pushing in the first marker placement portion 53 and the second marker placement portion 54 with a spear-like shape that has an outer diameter not as large as that of the third marker placement portion 55 but locally widens.

Note that the present invention is not limited to the embodiments described above, and various modifications may be made by those skilled in the art within the technical idea of the present invention. For example, as in another modification illustrated in FIG. 8, the maximum outer diameter D1 of the first marker placement portion 53 may be equal to or smaller than the outer diameter d1 of the marker-absent portion 52 on the proximal side with respect to the first marker placement portion 53. Although the reinforcing body 20 is disposed in the marker-absent portion 52 on the proximal side with respect to the first marker placement portion 53, the reinforcing body may not be disposed.

The detailed description above describes embodiments of a catheter used in a lumen such as a blood vessel. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A catheter comprising:
a shaft that has a lumen communicating from a distal end to a proximal end, the shaft having a plurality of markers that is radiopaque at different positions in an axial direction of the shaft;
the plurality of markers including a first marker that is a distal-most marker and a second marker that is located adjacent to the first marker on a proximal side; and
a thickness of the first marker along a radial direction of the shaft is different from a thickness of the second marker along the radial direction.

2. The catheter according to claim 1, wherein the thickness of the first marker along the radial direction is greater than the thickness of the second marker along the radial direction.

3. The catheter according to claim 1, wherein
one of the plurality of markers is a third marker disposed on the proximal side with respect to the second marker; and
the thickness of the first marker along the radial direction is greater than a thickness of the third marker along the radial direction.

4. The catheter according to claim 1, wherein
the thickness of the first marker along the radial direction is smaller than the thickness of the second marker along the radial direction.

5. The catheter according to claim 1, wherein
one of the plurality of markers is a third marker disposed on the proximal side with respect to the second marker; and
a thickness of the third marker along the radial direction is greater than the thickness of the first marker and the thickness of the second marker along the radial direction.

6. The catheter according to claim 1, wherein
the first marker on the distal-most side has a higher visibility with X-ray imaging than a visibility of the second marker with the X-ray imaging.

7. The catheter according to claim 3, wherein
the first marker on the distal-most side has a higher visibility with X-ray imaging than a visibility of the second marker and the third marker with the X-ray imaging.

8. The catheter according to claim 3, wherein
materials of the second marker and the third marker are less radiopaque than a material of the first marker.

9. The catheter according to claim 1, wherein
the shaft includes a plurality of marker placement portions in which the plurality of markers are placed and a plurality of marker-absent portions in which the plurality of markers are not placed in the axial direction, and wherein the plurality of marker placement portions and the plurality marker-absent portions are alternately arranged in the axial direction.

10. The catheter according to claim 9, wherein
an inner diameter of each of the plurality of marker placement portions is smaller than the inner diameter of the plurality of marker-absent portions.

11. The catheter according to claim 9, wherein
an outer diameter of a first marker placement portion where the first marker is placed is greater than an outer diameter of a marker-absent portion adjacent to the first marker placement portion.

12. The catheter according to claim 1, wherein
the shaft includes an inner layer defining an inner surface of the lumen, a reinforcing body disposed on the outer side of the inner layer in the radial direction, the plurality of markers disposed outside of the inner layer and the reinforcing body in the radial direction, and an outer layer formed on the outer side of the inner layer, the reinforcing body, and the plurality of markers in the radial direction.

13. The catheter according to claim 12, wherein
at least a part of the plurality of markers is embedded in the outer layer.

14. A catheter comprising:
a shaft that has a lumen communicating from a distal end to a proximal end, the shaft having a plurality of markers that is radiopaque at different positions in an axial direction of the shaft;
the plurality of markers includes a first marker that is a distal-most marker; and
an outer diameter of a first section of the shaft where the first marker is placed is different from an outer diameter of a section of the shaft where a marker included in the plurality of markers and different from the first marker is placed.

15. The catheter according to claim 14, wherein
the plurality of markers includes a second marker that is located adjacent to the first marker on a proximal side; and
the outer diameter of the first section is greater than an outer diameter of a second section of the shaft where the second marker is placed.

16. The catheter according to claim 14, wherein
one of the plurality of markers is a third marker located on the proximal side with respect to the second marker; and
the outer diameter of the first section is greater than an outer diameter of a third section of the shaft where the third marker is placed.

17. A catheter comprising:
a shaft that has a lumen communicating from a distal end to a proximal end, the shaft having a plurality of markers that is radiopaque at different positions in an axial direction of the shaft;
the plurality of markers including a first marker that is a distal-most marker and a second marker that is located adjacent to the first marker on a proximal side;
a thickness of the first marker along a radial direction of the shaft is different from a thickness of the second marker along the radial direction; and
an outer diameter of a first section of the shaft where the first marker is placed is different from an outer diameter of a section of the shaft where the second marker included in the plurality of markers is placed.

18. The catheter according to claim 17, wherein
the outer diameter of the first section is greater than an outer diameter of a second section of the shaft where the second marker is placed.

19. The catheter according to claim 17, wherein
one of the plurality of markers is a third marker located on the proximal side with respect to the second marker; and
the outer diameter of the first section is greater than an outer diameter of a third section of the shaft where the third marker is placed.

20. The catheter according to claim 17, wherein
one of the plurality of markers is a third marker disposed on the proximal side with respect to the second marker; and
the thickness of the first marker along the radial direction is greater than a thickness of the third marker along the radial direction.
